(19) United States
(12) Patent Application Publication
(10) Pub. No.: US 2012/0183953 A1
(43) Pub. Date: Jul. 19, 2012

(54) GENOME ASSEMBLY

(75) Inventors: Nianqing XIAO, Rockville, MD (US); John K. HENKHAUS, Washington, DC (US); Bin ZHU, Gaithersburg, MD (US); Deacon SWEENEY, Gaithersburg, MD (US); Thomas ANANTHARAMAN, Gaithersburg, MD (US); Ryan PTASHKIN, Bethesda, MD (US)

(73) Assignee: OpGen, Inc., Gaithersburg, MD (US)

(21) Appl. No.: 13/096,408

(22) Filed: Apr. 28, 2011

Related U.S. Application Data
(60) Provisional application No. 61/432,828, filed on Jan. 14, 2011.

(51) Int. Cl. C12Q 1/68; C12P 19/34

(52) U.S. Cl. 435/6.1; 435/91.5

(57) ABSTRACT

The invention generally relates to methods for assembling sequence contigs. In certain embodiments, methods of the invention involve converting sequence contigs into maps, generating a plurality of single molecule restriction maps, aligning single molecule restriction maps to ends of the maps of the sequence contigs, thereby producing extended sequence contigs, and aligning extended sequence contigs.

### RELATED APPLICATION

The present application claims the benefit of and priority to U.S. provisional application Ser. No. 61/432,828, filed Jan. 14, 2011, the content of which is incorporated by reference herein in its entirety.

### FIELD OF THE INVENTION

The invention generally relates to methods for assembling sequence contigs.

### BACKGROUND

Methods to sequence or identify significant fractions of the human genome and genetic variations within those segments are becoming commonplace. However, a major impediment to understanding health implications of variations found in every human being remains unraveling of the functional meaning of all sequence differences in every individual. Whole genome sequencing is an important first step that will allow geneticists and physicians to develop a full functional understanding of that data.

Whole genome sequencing generally involves randomly breaking up DNA into numerous small segments that are sequenced to obtain reads. Multiple overlapping reads for the target DNA are obtained by performing several rounds of this fragmentation and sequencing. Computer programs then use the overlapping ends of different reads to assemble them into sequence contigs. Sequence contigs are then assembled to obtain a continuous sequence.

Whole genome sequencing projects typically produce hundreds or thousands of relatively short sequence contigs. The contigs typically cover a majority of an organism's genome but their relative order and orientation is difficult to determine because there are gaps between the contigs that must be filled. In practice, whole genome sequencing uses enormous amounts of information that is rife with ambiguities and sequencing errors. Assembly of complex genomes is additionally complicated by the great abundance of repetitive sequence in a genome, meaning similar short sequence reads could come from completely different parts of the sequence. Many overlapping reads for each segment of the original DNA are necessary to overcome these difficulties and accurately assemble the sequence. For example, to complete the Human Genome Project, most of the human genome was sequenced at 12× or greater coverage; that is, each base in the final sequence was present, on average, in 12 reads. Even so, current methods have failed to isolate or assemble a reliable sequence for approximately 1% of the (euchromatic) human genome.

### SUMMARY

Methods of the invention use mapping, particularly optical mapping, to simplify the process of sequence contig assembly (e.g., ordering and orientation of contigs). Optical mapping can produce ordered restriction maps by using fluorescence microscopy to visualize restriction endonuclease cutting events on individual labeled DNA molecules. Methods of the invention involve converting obtained sequence contigs into maps. Additionally, long strands of nucleic acids are extracted from a sample and single molecule maps are generated from the long strands of nucleic acids. The single molecule maps are aligned with ends of the map of the sequence contig, thereby producing extended sequence contigs. Generally, the extended sequence contigs bridge gaps that previously existed among unextended sequence contigs. The extended sequence contigs are then aligned with each other to produce a continuous sequence. In certain embodiments, the maps are optical maps.

Methods of the invention take advantage of the fact that optical mapping uses long strands of nucleic acid (e.g., several hundred kb). The use of long nucleic acid strands allow optical mapping to span gaps between sequence contigs that are difficult to cover with short sequence reads. Thus, methods of the invention generate data in regions of the genome that often present difficulty for sequencing reactions. This data can bridge the gap between sequence contigs and can be used to ensure proper ordering and orientation of the sequence contigs. Methods of the invention dramatically reduce costs and time associated with sequencing projects.

Methods of the invention may be used with any sequencing project and may be used in conjunction with sequencing of human DNA or DNA from other organisms, such as microorganisms (e.g., a bacterium, a fungus, or a virus).

In other aspects, the invention provides methods for assembling sequence contigs that involve using optical mapping to generate single molecule restriction maps, extending sequence contigs by aligning single molecule restriction maps to ends of optical maps of the sequence contigs, and aligning the extended sequence contigs.

### DETAILED DESCRIPTION

The invention generally relates to methods for assembling sequence contigs. Methods of the invention involve converting obtained sequence contigs into maps. Additionally, long strands of nucleic acids are extracted from a sample and single molecule maps are generated from the long strands of nucleic acids. The single molecule maps are aligned with ends of the map of the sequence contig, thereby producing extended sequence contigs. Generally, the extended sequence contigs bridge gaps that previously existed among unextended sequence contigs. The extended sequence contigs are then aligned with each other to produce a continuous sequence. In certain embodiments, the maps are optical maps.

The following sections discuss general considerations for sample nucleic acids, nucleic acid sequencing, mapping (particularly optical mapping), contig extension and alignment of extended sequence contigs.

### Sample Nucleic Acids

Nucleic acids include deoxyribonucleic acid (DNA) and/or ribonucleic acid (RNA). Nucleic acids can be synthetic or derived from naturally occurring sources. In one embodiment, nucleic acids are isolated from a biological sample containing a variety of other components, such as proteins, lipids and non-sample nucleic acids. Nucleic acids can be obtained from any cellular material, obtained from a human or other mammal, plant, or microorganism (e.g., bacterium, fungus, virus or any other cellular organism). In certain embodiments, the nucleic acids are obtained from a single cell. Biological samples for use in the present invention include viral particles or preparations. Nucleic acids can be obtained directly from an organism or from a biological sample obtained from an organism, e.g., from blood, urine, cerebrospinal fluid, seminal fluid, saliva, sputum, stool and tissue. Any tissue or body fluid specimen may be used as a source for nucleic acid for use in the invention. Nucleic acids can also be isolated from cultured cells, such as a primary cell culture or a cell line. The cells or tissues from which nucleic acids are obtained can be infected with a virus or other intracellular pathogen. A sample can also be total RNA extracted from a biological specimen, a cDNA library, viral, or genomic DNA.

Nucleic acid obtained from biological samples typically is fragmented to produce suitable fragments for analysis. In one embodiment, nucleic acid from a biological sample is fragmented by sonication. Nucleic acids can be obtained as described in U.S. Patent Application Publication Number US2002/0190663 A1, published Oct. 9, 2003. Generally, nucleic acid can be extracted from a biological sample by a variety of techniques such as those described by Maniatis, et al., Molecular Cloning: A Laboratory Manual, Cold Spring Harbor, N.Y., pp. 280-281 (1982). Generally, individual nucleic acids can be from about 5 bases to about 20 kb. Nucleic acid molecules may be single-stranded, double-stranded, or double-stranded with single-stranded regions (for example, stem- and loop-structures).

A biological sample as described herein may be homogenized or fractionated in the presence of a detergent or surfactant. The concentration of the detergent in the buffer may be about 0.05% to about 10.0%. The concentration of the detergent can be up to an amount where the detergent remains soluble in the solution. In a preferred embodiment, the concentration of the detergent is between 0.1% to about 2%. The detergent, particularly a mild one that is nondenaturing, can act to solubilize the sample. Detergents may be ionic or nonionic. Examples of nonionic detergents include triton, such as the Triton® X series (Triton® X-100 t-Oct-C₆H₄—(OCH₂—CH₂)ₓOH, x=9-10, Triton® X-100R, Triton® X-114 x=7-8), octyl glucoside, polyoxyethylene(9)dodecyl ether, digitonin, IGEPAL® CA630 octylphenyl polyethylene glycol, n-octyl-beta-D-glucopyranoside (betaOG), n-dodecyl-beta, Tween® 20 polyethylene glycol sorbitan monolaurate, Tween® 80 polyethylene glycol sorbitan monooleate, polidocanol, n-dodecyl beta-D-maltoside (DDM), NP-40 nonylphenyl polyethylene glycol, C12E8 (octaethylene glycol n-dodecyl monoether), hexaethyleneglycol mono-n-tetradecyl ether (C14EO6), octyl-beta-thioglucopyranoside (octyl thioglucoside, OTG), Emulgen, and polyoxyethylene 10 lauryl ether (C12E10). Examples of ionic detergents (anionic or cationic) include deoxycholate, sodium dodecyl sulfate (SDS), N-lauroylsarcosine, and cetyltrimethylammoniumbromide (CTAB). A zwitterionic reagent may also be used in the purification schemes of the present invention, such as Chaps, zwitterion 3-14, and 3-[(3-cholamidopropyl)dimethylammonio]-1-propanesulf-onate. It is contemplated also that urea may be added with or without another detergent or surfactant.

Lysis or homogenization solutions may further contain other agents, such as reducing agents. Examples of such reducing agents include dithiothreitol (DTT), .beta.-mercaptoethanol, DTE, GSH, cysteine, cysteamine, tricarboxyethyl phosphine (TCEP), or salts of sulfurous acid.

### Nucleic Acid Sequencing

Any sequencing method known in the art e.g., ensemble sequencing or single molecule sequencing, may be used with methods of the invention. One conventional method to perform sequencing is by chain termination and gel separation, as described by Sanger et al., Proc Natl Acad Sci USA, 74 (12): 5463 67 (1977). Another conventional sequencing method involves chemical degradation of nucleic acid fragments. See, Maxam et al., Proc. Natl. Acad. Sci., 74: 560 564 (1977). Finally, methods have been developed based upon sequencing by hybridization. See, e.g., Drmanac, et al., Nature Biotech., 16: 54 58 (1998). The content of each reference is incorporated by reference herein in its entirety.

In certain embodiments, sequencing is performed by the Sanger sequencing technique. Classical Sanger sequencing involves a single-stranded DNA template, a DNA primer, a DNA polymerase, radioactively or fluorescently labeled nucleotides, and modified nucleotides that terminate DNA strand elongation. If the label is not attached to the dideoxynucleotide terminator (e.g., labeled primer), or is a monochromatic label (e.g., radioisotope), then the DNA sample is divided into four separate sequencing reactions, containing four standard deoxynucleotides (dATP, dGTP, dCTP and dTTP) and the DNA polymerase. To each reaction is added only one of the four dideoxynucleotides (ddATP, ddGTP, ddCTP, or ddTTP). These dideoxynucleotides are the chain-terminating nucleotides, lacking a 3′-OH group required for the formation of a phosphodiester bond between two nucleotides during DNA strand elongation. If each of the dideoxynucleotides carries a different label, however, (e.g., 4 different fluorescent dyes), then all the sequencing reactions can be carried out together without the need for separate reactions.

Incorporation of a dideoxynucleotide into the nascent, i.e., elongating, DNA strand terminates DNA strand extension, resulting in a nested set of DNA fragments of varying length. Newly synthesized and labeled DNA fragments are denatured, and separated by size using gel electrophoresis on a denaturing polyacrylamide-urea gel capable of resolving single-base differences in chain length. If each of the four DNA synthesis reactions was labeled with the same, monochromatic label (e.g., radioisotope), then they are separated in one of four individual, adjacent lanes in the gel, in which each lane in the gel is designated according to the dideoxynucleotide used in the respective reaction, i.e., gel lanes A, T, G, C. If four different labels were utilized, then the reactions can be combined in a single lane on the gel. DNA bands are then visualized by autoradiography or fluorescence, and the DNA sequence can be directly read from the X-ray film or gel image.

The terminal nucleotide base is identified according to the dideoxynucleotide that was added in the reaction resulting in that band or its corresponding direct label. The relative positions of the different bands in the gel are then used to read (from shortest to longest) the DNA sequence as indicated. The Sanger sequencing process can be automated using a DNA sequencer, such as those commercially available from PerkinElmer, Beckman Coulter, Life Technologies, and others.

In other embodiments, sequencing of the nucleic acid is accomplished by a single-molecule sequencing by synthesis technique. Single molecule sequencing is shown for example in Lapidus et al. (U.S. Pat. No. 7,169,560), Quake et al. (U.S. Pat. No. 6,818,395), Harris (U.S. Pat. No. 7,282,337), Quake et al. (U.S. patent application number 2002/0164629), and Braslaysky, et al., PNAS (USA), 100: 3960-3964 (2003), the contents of each of these references is incorporated by reference herein in its entirety. Briefly, a single-stranded nucleic acid (e.g., DNA or cDNA) is hybridized to oligonucleotides attached to a surface of a flow cell. The oligonucleotides may be covalently attached to the surface or various attachments other than covalent linking as known to those of ordinary skill in the art may be employed. Moreover, the attachment may be indirect, e.g., via a polymerase directly or indirectly attached to the surface. The surface may be planar or otherwise, and/or may be porous or non-porous, or any other type of surface known to those of ordinary skill to be suitable for attachment. The nucleic acid is then sequenced by imaging the polymerase-mediated addition of fluorescently-labeled nucleotides incorporated into the growing strand surface oligonucleotide, at single molecule resolution.

Other single molecule sequencing techniques involve detection of pyrophosphate as it is cleaved from incorporation of a single nucleotide into a nascent strand of DNA, as is shown in Rothberg et al. (U.S. Pat. Nos. 7,335,762, 7,264,929, 7,244,559, and 7,211,390) and Leamon et al. (U.S. Pat. No. 7,323,305), the contents of each of which is incorporated by reference herein in its entirety.

If the nucleic acid from the sample is degraded or only a minimal amount of nucleic acid can be obtained from the sample, PCR can be performed on the nucleic acid in order to obtain a sufficient amount of nucleic acid for sequencing (See e.g., Mullis et al. U.S. Pat. No. 4,683,195, the contents of which are incorporated by reference herein in its entirety).

### Data Analysis

Alignment and/or compilation of sequence results obtained can be performed by methods known in the art using commercially available software programs. For example, Flicek et al. (Nature Methods 6:S6-S12, 2009) describes several algorithmic approaches that align or assemble sequence reads into sequence contigs and then align sequence contigs. See also Kent (Genome Res. 2002, 12 (4):656-664, 2002), Smith et al. (Mol Biol., 147:195-197, 1981), Pearson et al. (Proc Natl Acad Sci, 85:2444-2448, 1988), Altschul et al. (J Mol Biol., 215:403-410, 1990), Altschul et al. (Nucleic Acids Res., 25:3389-3402, 1997), Zhang et al. (J Comput Biol., 7:203-214, 2000), Gish et al. (Nat Genet., 3:266-272, 1993), States (J Comput Biol., 1:39-50, 1994), Florea et al. (Genome Res., 8:967-974, 1998), Karplus et al. (Bioinformatics, 14:846-856, 1998), Gotch et al. (Bull Math Biol., 52:359-373, 1990), Gotch et al. (Bioinformatics, 16:190-202, 2000), and Ning et al. (Genome Res., 11:1725-1729, 2001). The content of each of these references is incorporated herein in its entirety.

Commercially available software programs include BLAST (local search with fast k-tuple heuristic (Basic Local Alignment Search Tool)), FASTA (local search with fast k-tuple heuristic), GGSEARCH/GLSEARCH (Global:Global (GG), Global:Local (GL) alignment with statistics), HMMER (local and global search with profile Hidden Markov models), HHpred/HHsearch (pairwise comparison of profile Hidden Markov models), IDF (Inverse Document Frequency), PSI-BLAST (position-specific iterative BLAST, local search with position-specific scoring matrices), SAM (local and global search with profile Hidden Markov models), SSEARCH (Smith-Waterman search), ACT (Synteny and comparative genomics), AVID (Pairwise global alignment with whole genomes), Mauve (Multiple alignment of rearranged genomes), MGA (Multiple Genome Aligner), Mulan (Local multiple alignments of genome-length sequences), Multiz (Multiple alignment of genomes), Sequerome (Profiling sequence alignment data with major servers/services), Sequilab (Profiling sequence alignment data from NCBI-BLAST results with major servers/services), Shuffle-LAGAN (Pairwise glocal alignment of completed genome regions), and SIBsim4/Sim4 (align an expressed DNA sequence with a genomic sequence, allowing for introns).

### Optical Mapping

A problem associated with sequence assembly is alignment and orientation of sequence contigs. The contigs typically cover a majority of an organism's genome but their relative order and orientation is difficult to determine because there are gaps between the contigs that must be filled. Methods of the invention use optical mapping to simplify the process of sequence contig assembly (e.g., ordering and orientation of contigs). Methods of the invention take advantage of the fact that optical mapping uses long strands of nucleic acid (e.g., several hundred kb). The use of long nucleic acid strands allow optical mapping to span gaps between sequence contigs that are difficult to cover with short sequence reads. Thus, methods of the invention generate data in regions of the genome that often present difficulty for sequencing reactions. This data can bridge the gap between sequence contigs and can be used to ensure proper ordering and orientation of the sequence contigs.

Optical mapping is a single-molecule technique for production of ordered restriction maps from a single DNA molecule (Samad et al., Genome Res. 5:1-4, 1995). During some applications, individual fluorescently labeled DNA molecules are elongated and fixed on the surface using methods of the invention. The added endonuclease cuts the DNA at specific points, and the fragments are imaged. Id. Exemplary endonucleases include BglII, NcoI, XbaI, and BamHI. Exemplary combinations of restriction enzymes include:

| | | |
|---|---|---|
| AflII | ApaLI | BglII |
| AflII | BglII | NcoI |
| ApaLI | BglII | NdeI |
| AflII | BglII | MluI |
| AflII | BglII | PacI |
| AflII | MluI | NdeI |
| BglII | NcoI | NdeI |
| AflII | ApaLI | MluI |
| ApaLI | BglII | NcoI |
| AflII | ApaLI | BamHI |
| BglII | EcoRI | NcoI |
| BglII | NdeI | PacI |
| BglII | Bsu36I | NcoI |
| ApaLI | BglII | XbaI |
| ApaLI | MluI | NdeI |
| ApaLI | BamHI | NdeI |
| BglII | NcoI | XbaI |
| BglII | MluI | NcoI |
| BglII | NcoI | PacI |
| MluI | NcoI | NdeI |
| BamHI | NcoI | NdeI |
| BglII | PacI | XbaI |
| MluI | NdeI | PacI |
| Bsu36I | MluI | NcoI |
| ApaLI | BglII | NheI |
| BamHI | NdeI | PacI |
| BamHI | Bsu36I | NcoI |
| BglII | NcoI | PvuII |
| BglII | NcoI | NheI |
| BglII | NheI | PacI |

Restriction maps can be constructed based on the number of fragments resulting from the digest. Id. Generally, the final map is an average of fragment sizes derived from similar molecules. Id.

Optical mapping and related methods are described in U.S. Pat. No. 5,405,519, U.S. Pat. No. 5,599,664, U.S. Pat. No. 6,150,089, U.S. Pat. No. 6,147,198, U.S. Pat. No. 5,720,928, U.S. Pat. No. 6,174,671, U.S. Pat. No. 6,294,136, U.S. Pat. No. 6,340,567, U.S. Pat. No. 6,448,012, U.S. Pat. No. 6,509,158, U.S. Pat. No. 6,610,256, and U.S. Pat. No. 6,713,263. All the cited patents are incorporated by reference herein in their entireties.

Optical Maps are constructed as described in Reslewic et al., Appl Environ Microbiol. 2005 September; 71 (9):5511-22, incorporated by reference herein. Briefly, individual chromosomal fragments from test organisms are immobilized on derivatized glass by virtue of electrostatic interactions between the negatively-charged DNA and the positively-charged surface, digested with one or more restriction endonuclease, stained with an intercalating dye such as YOYO-1 (Invitrogen) and positioned onto an automated fluorescent microscope for image analysis. Since the chromosomal fragments are immobilized, the restriction fragments produced by digestion with the restriction endonuclease remain attached to the glass and can be visualized by fluorescence microscopy, after staining with the intercalating dye. The size of each restriction fragment in a chromosomal DNA molecule is measured using image analysis software and identical restriction fragment patterns in different molecules are used to assemble ordered restriction maps covering the entire chromosome.

Methods of the invention involve converting obtained sequence contigs into optical maps. Additionally, long strands of nucleic acids are extracted from a sample and single molecule optical maps are generated from the long strands of nucleic acids. The single molecule optical maps are aligned with ends of the optical map of the sequence contig, thereby producing extended sequence contigs. Generally, the extended sequence contigs bridge gaps that previously existed among unextended sequence contigs. The extended sequence contigs are then aligned with each other to produce a continuous sequence.

Map alignments between single molecule optical maps and optical maps of the sequence contigs are generated with a dynamic programming algorithm that finds the optimal alignment of two restriction maps according to a scoring model that incorporates fragment sizing errors, false and missing cuts, and missing small fragments (See Myers et al, Bull Math Biol 54:599-618 (1992); Tang et al, J Appl Probab 38:335-356 (2001); and Waterman et al., Nucleic Acids Res 12:237-242). For a given alignment, the score is proportional to the log of the length of the alignment, penalized by the differences between the two maps, such that longer, better-matching alignments will have higher scores.

To generate extended sequence contigs, each single molecule optical map is aligned against the optical maps of the sequence contigs. From these alignments, a pair-wise alignment analysis is performed to determine “percent dissimilarity” between the single molecule optical maps and the optical maps of the sequence contigs taking the total length of the unmatched regions in both maps divided by the total size of both maps. These dissimilarity measurements are used as inputs into the agglomerative clustering method “Agnes” as implemented in the statistical package “R”. Briefly, this clustering method works by initially placing each entry in its own space, then iteratively joining the single molecule optical map to the optical map of the sequence contig that most closely matches that single molecule optical map, thereby producing extended sequence contigs. Generally, the extended sequence contigs bridge gaps that previously existed among unextended sequence contigs, and thus generate regions of overlap between the extended sequence contigs, allowing for their alignment and joining to form a continuous sequence. The process is then repeated for aligning of the extended sequence contigs.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein.

What is claimed is:

1. A method for assembling sequence contigs, the method comprising:
converting sequence contigs into maps;
generating a plurality of single molecule restriction maps;
aligning single molecule restriction maps to ends of the maps of the sequence contigs, thereby producing extended sequence contigs; and
aligning extended sequence contigs.

2. The method according to claim 1, wherein generating comprises:
introducing the nucleic acids to a charged substrate so that the nucleic acids become elongated and fixed on the subject in a manner in which the nucleic acids remain accessible for enzymatic reactions;
digesting the nucleic acids enzymatically to produce one or more restriction digests; and
constructing a map from the restriction digests.

3. The method according to claim 2, wherein the substrate is derivatized glass.

4. The method according to claim 3, wherein the glass is derivatized with silanes.

5. The method according to claim 1, wherein the sample is a human tissue or body fluid.

6. The method according to claim 1, wherein the sample is from a microorganism.

7. The method according to claim 6, wherein the microorganism is a selected from the group consisting of a bacterium, a fungus, and a virus.

8. The method according to claim 1, further comprising determining contig arrangement.

9. The method according to claim 1, further comprising determining contig orientation.

10. The method according to claim 1, further comprising identifying assembly errors in the sequence contigs.

11. The method according to claim 1, wherein the nucleic acids are several hundred kilobases in length.

12. The method according to claim 1, wherein the single molecule restriction maps span gaps between the sequence contigs.

13. A method for assembling sequence contigs, the method comprising:
using mapping to generate single molecule restriction maps;
extending sequence reads by aligning single molecule restriction maps to ends of maps of sequence contigs, thereby producing extended sequence contigs; and
aligning the extended sequence contigs.

14. The method according to claim 13, wherein generating comprises:
introducing the nucleic acids to a charged substrate so that the nucleic acids become elongated and fixed on the subject in a manner in which the nucleic acids remain accessible for enzymatic reactions;
digesting the nucleic acids enzymatically to produce one or more restriction digests; and
constructing a map from the restriction digests.

15. The method according to claim 14, wherein the substrate is derivatized glass.

16. The method according to claim 15, wherein the glass is derivatized with silanes.

17. The method according to claim 14, wherein the sample is a human tissue or body fluid.

18. The method according to claim 14, wherein the sample is from a microorganism.

19. The method according to claim 18, wherein the microorganism is a selected from the group consisting of a bacterium, a fungus, and a virus.

20. The method according to claim 14, further comprising determining contig arrangement.

21. The method according to claim 14, further comprising determining contig orientation.

22. The method according to claim 14, further comprising identifying assembly errors in the sequence contigs.

23. The method according to claim 14, wherein the single molecule restriction maps span gaps between the sequence contigs.
